(19) United States
(12) Patent Application Publication
(10) Pub. No.: US 2014/0307258 A1
(43) Pub. Date: Oct. 16, 2014

(54) OPTICAL ANALYTE MEASUREMENT

(71) Applicants: Shane Kirkbride, Colorado Springs, CO (US); Terrance E. Boult, Monument, CO (US)

(72) Inventors: Shane Kirkbride, Colorado Springs, CO (US); Terrance E. Boult, Monument, CO (US)

(73) Assignee: Methode Electronics, Inc., Carthage, IL (US)

(21) Appl. No.: 13/809,325

(22) PCT Filed: Jul. 11, 2011
(86) PCT No.: PCT/US11/43549

§ 371 Date: Dec. 9, 2013

Related U.S. Application Data
(60) Provisional application No. 61/362,914, filed on Jul. 9, 2010.

(51) Int. Cl. G01N 21/25; A61B 5/00; A61B 5/1455

(52) U.S. Cl. CPC G01N 21/255; A61B 5/1455; A61B 5/4845
USPC 356/300; 600/314

(57) ABSTRACT

Ethanol is non-invasively tested using temperature-modulated spectroscopy (TMS). The TMS approach uses the active control of temperature to vary the spectral response of the IR-LED output, effectively sliding a spectral pulse across the ethanol sample, revealing the peaks and valleys of ethanol's spectral response.

### REFERENCE TO RELATED APPLICATION

The present application claims the benefit of U.S. Provisional Patent Application No. 61/362,914, filed Jul. 9, 2010. Related information is disclosed in U.S. Provisional Patent Application No. 61/362,922, filed Jul. 9, 2010. The disclosures of the above applications are hereby incorporated by reference in their entireties into the present disclosure.

### FIELD OF THE INVENTION

The present invention is directed to measurement of an analyte, such as ethanol, and more particularly to non-invasive, in vivo optical measurement of such an analyte.

### DESCRIPTION OF RELATED ART

Blood alcohol content (BAC), also called blood alcohol concentration, blood ethanol concentration, or blood alcohol level, is most commonly used as a metric of alcohol intoxication for legal or medical purposes. Blood alcohol tests have a flaw in that they assume that the person being tested is average in various ways.

For example, on average the ratio of blood alcohol content to breath alcohol content (the partition ratio) is 2100 to 1. In other words, there are 2100 parts of alcohol in the blood for every part in the breath. However, the actual ratio in any given individual can vary from 1300:1 to 3100:1, or even more widely. This ratio varies not only from person to person, but within one person from moment to moment. Thus a person with a true blood alcohol level of 0.08 but a partition ratio of 1700:1 at the time of testing would have a 0.10 reading on a Breathalyzer calibrated for the average 2100:1 ratio.

A similar assumption is made in urinalysis. When urine is analyzed for alcohol, the assumption is that there are 1.3 parts of alcohol in the urine for every 1 part in the blood, even though the actual ratio can vary greatly.

Breath alcohol testing further assumes that the test is post-absorptive—that is, that the absorption of alcohol in the subject's body is complete. If the subject is still actively absorbing alcohol, their body has not reached a state of equilibrium where the concentration of alcohol is uniform throughout the body. Most forensic alcohol experts reject test results during this period as the amounts of alcohol in the breath will not accurately reflect a true concentration in the blood.

U.S. Patent Application Publication No. 2006/0002598 teaches a noninvasive alcohol sensor. An illumination subsystem provides light at discrete wavelengths to a skin site of an individual. A detection subsystem receives light scattered from the skin site. A computational unit is interfaced with the detection system. The computational unit has instructions for deriving a spatially distributed multispectral image from the received light at the discrete wavelengths. The computational unit also has instructions for comparing the derived multispectral image with a database of multispectral images to identify the individual.

The illumination subsystem may comprise a light source that provides the light to the plurality of discrete wavelengths and illumination optics to direct the light to the skin site. In some instances, a scanner mechanism may also be provided to scan the light in a specified pattern. The light source may comprise a plurality of quasi-monochromatic light sources, such as LEDs or laser diodes. Alternatively, the light source may comprise a broadband light source, such as an incandescent bulb or glowbar, and a filter disposed to filter light emitted from the broad band source. The filter may comprise a continuously variable filter in one embodiment. In some cases, the detection system may comprise a light detector, an optically dispersive element, and detection optics. The optically dispersive element is disposed to separate wavelength components of the received light, and the detection optics direct the received light to the light detector. In one embodiment, both the illumination and detection subsystems comprise a polarizer. The polarizers may be circular polarizers, linear polarizers, or a combination. In the case of linear polarizers, the polarizers may be substantially crossed relative to each other.

However, it would be desirable to provide a simpler and more compact way of achieving the same result.

### SUMMARY OF THE INVENTION

It is therefore an object of the invention to provide such a simpler and more compact way for optical detection of an analyte such as ethanol.

To achieve the above and other objects, the present invention is directed to a technique called temperature-modulated spectrometry (TMS). The development of TMS included an investigation into the relationship between the spectral response of infrared light emitting diodes (IR-LEDs) and temperature. The TMS approach uses the active control of temperature to vary the spectral response of the IR-LED output, effectively sliding a spectral pulse across the ethanol sample, revealing the peaks and valleys of ethanol's spectral response. TMS can be used in any other field of endeavor using spectroscopic analysis.

Throughout the present specification, the term “LED” is meant to include other semiconductor emitters such as laser diodes, vertical cavity surface emitting lasers, and other such devices.

A simulation of the TMS approach was created using COTS IR-LEDs with a spectral response in the region of 2.1 μm-2.50 μm. The TMS approach will yield a low cost, compact system with very few parts (no moving parts). The estimated resulting unit cost is less than $50 per unit in low volume (thousands) and less than $15 in higher volumes.

Initial research was done to do first order approximations of the signal to noise ratio (SNR) of actual components, and to affirm the repeatability of the temperature modulation in the IR-LED. An experimental setup was constructed using IR-LEDS to show the peaks and valleys of the differentiated spectral response of various concentrations of ethanol (95%, 40%, 0.15% and 0.015%) and tap water. The IR-LED was modulated to sweep across 2.3 μm, 2.32 μm, 2.35 μm and 2.38 μm and clearly demonstrated that the measurement of ethanol, even at small levels, is possible with this approach.

This is a sensitive procedure requiring careful environmental control in order to get the precise measurement necessary for an accurate reading. However, even with a rudimentary experimental apparatus, the peaks and valleys in ethanol at 0.015% ethanol or 15 mg ethanol/di water are measurable.

The TMS approach disclosed herein effectively detects ethanol in mixture down to 0.015% (in tap water). This new spectroscopic approach has great promise.

### BRIEF DESCRIPTION OF THE DRAWINGS

A preferred embodiment of the present invention will be set forth in detail with reference to the drawings, in which:

FIG. 1 shows final experimental results;

FIG. 2 is a manufacturer's specification sheet showing spectral response changing as wavelength changes;

FIG. 3 shows individual LED and photodiode components and their drivers;

FIG. 4 shows an initial horizontal prototype allowing for an measurement of path length and adjustments to alignment and intensity;

FIG. 5 is a screen shot of the oscilloscope measuring the output of the photodiode driver;

FIG. 6 shows raw (left) and sorted (right) data from the oscilloscope and shows how the photodiode amplifier clamps the signal at a little over 0.5 v;

FIG. 7 shows measurements from LED23a showing the response across the temperatures sweep with no medium present; this is the raw data taken from the photodiode and is unsmoothed;

FIG. 8 shows raw unsmoothed measurements from LED23a showing the response of 95% ethanol and tap water as a function of temperature and shows a shift in the vertical axis;

FIG. 9 shows three runs of calculated absorbance for ethanol and water, showing the range of variations seen;

FIG. 10 shows the mean of ethanol and water, with dashed lines showing one standard deviation;

FIG. 11 shows derivatives of the mean of ethanol and water with dashed lines showing the standard deviation;

FIG. 12 shows normalized absorbance for ethanol and water (top left) and 40/60 ethanol and water (top right) and shows normalized data with absorbance measurement of water subtracted from absorbance measurements of 15 mg/dl and 150 mg/dl;

FIG. 13 shows the derivative of calculated absorbance for ethanol and water (top right) clearly showing peaks and valleys in ethanol;

FIG. 14 shows a wrap-around error that can occur if the temperature of the LED is still increasing but the temperature on the sensor has started decreasing;

FIG. 15 shows how the temperature can be as much as 3 degrees off for some measurements;

FIG. 16 shows the estimated response of ethanol with the measured the peaks and troughs labeled, in which the area swept starts at approximately 2.3 μm and goes until 2.37 μm;

FIG. 17 shows (left) the spectral absorbance of ethanol verses tap water; the scale on the right y-axis corresponds to ethanol, and the scale on the left y-axis corresponds to tap water; the right graph is zoomed around the region of interest for LED23 in which ethanol has a valley, peak and valley;

FIG. 18 shows (left) the expected response from LED23a when the temperature varies (wavelengths 2290 to 2360) and (right) the simulated photodetector response induced by a temperature sweep; the broader peak in the LED illumination smooths out the smaller peaks/valleys in the expected response of water and reduces the amplitude of the peaks/valleys in ethanol; the simulated response in water shows more increase in absorbance at higher wavelength than the measurements;

FIG. 19 shows the estimated absorbance difference from water in mixtures of ethanol and water;

FIG. 20 shows (left) the derivative of absorbency for ethanol and water from FIG. 19 and (right) the derivative of the differenced absorbencies for mixtures shown in FIG. 19;

FIG. 21 shows various elements and there effective wavelengths in relation to semiconductor properties;

FIG. 22 shows the energy shifts and temperature changes causing a shift in wavelength;

FIG. 23 shows the relationship between the light source and its critical angles along with their relationship to reflection and transmission;

FIG. 24 shows the basic structure of an SLED (left) and an ELED (right);

FIG. 25 shows the difference in spectral response for the SLED vs. the ELED; SLEDs have a more distributed relative output power, while the energy in ELEDs is more concentrated;

FIG. 26 shows the spectral response of ethanol;

FIG. 27 shows the chemical structure of hemoglobin;

FIG. 28 shows the optical paths light takes through the skin;

FIG. 29 shows the spectral remittance of the dermis at various wavelengths;

FIG. 30 shows blanched and unbalanced results for lighter skin and darker skin and shows that the responses seem to be converging at the longer wavelengths;

FIG. 31 shows (left) basic tissue anatomy and (right) four different LED/photodiode placements;

FIG. 32 shows an example of how the optimal configuration of the LED/photodiode can be configured;

FIG. 33 shows a Czerny Turner dispersion element with an area sensor;

FIG. 34 shows beam-splitting the LED illumination to improve normalization; and

FIG. 35 is a block diagram of a system implementing the preferred or another embodiment.

### DETAILED DESCRIPTION OF THE PREFERRED EMBODIMENT

A preferred embodiment will be set forth in detail with reference to the drawings, in which like reference numerals refer to like elements or steps throughout.

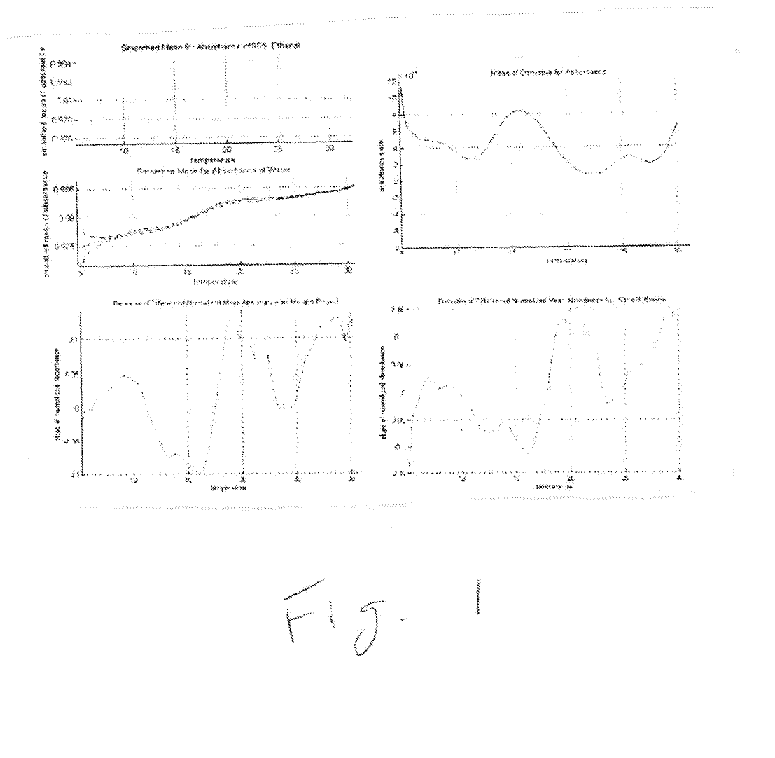

FIG. 1 displays the final experimental results. Measured temperatures correspond to approximations of measured wavelengths, 2.29 μm-2.36 μm, in which ethanol has a distinctive valley/peak/valley pattern. Sweeping through this region, water has a predominately increasing absorbance. Mixtures have features that are more complex than either element. The zero crossings show a clear presence of ethanol even at 0.015% ethanol using the TMS processes.

More specifically, in FIG. 1, the upper left shows measured absorbance and standard deviations for ethanol and water. The peaks and valleys in ethanol are visible/measurable. Upper right shows the derivative of absorbance where the peaks and valleys correspond to locations of zero of the derivative. Because water is constantly increasing, any ethanol and water mixture will not have the same peaks and valleys. The data was processed as described in this report. The lower two plots show derivatives for 15 mg/dl (0.015%) ethanol on the lower left and 150 mg/dl (0.15%) ethanol in the lower right. In both mixtures, the first two ethanol zero crossings (valley/peak) are still visible in the mixtures thought somewhat shifted as the increasing water absorbance impacts their location. The noise in processing mixture introduces two new zero crossings and shifts the rightmost valley in ethanol from 29 degrees down to 25 degrees.

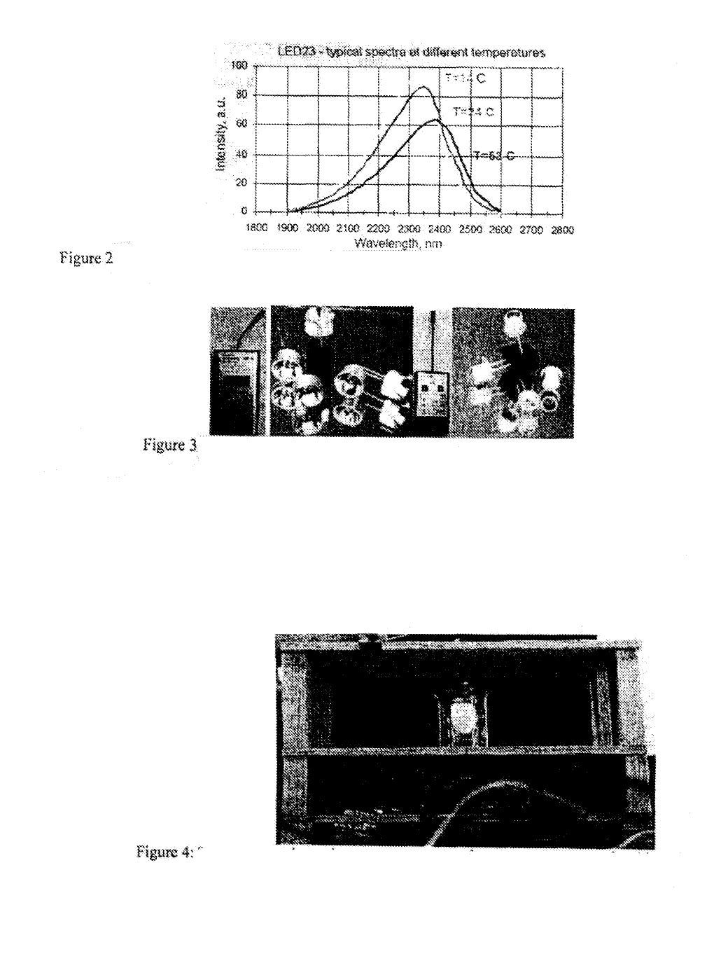

The key insight behind the proposed solution is that LEDs and solid state lasers generate photons depending on temperature, which shift the spectral range and multitude. This dependence has led to dozens of patents and processes using temperature to stabilize the LED or IR laser's output. As temperature increases, the LED's overall wavelength distribution value shifts to longer wavelengths. Additionally, the overall efficiency of the radiation decreases, as shown in the spectral curves in FIG. 2. This temperature dependence is normally considered a problem causing variations in LED output. Typically, system designers address the temperature dependence by stabilizing the temperature of the LED. Micro shifts in the spectral outputs occur as a result of this temperature dependence; a feature effectively allowing us to obtain spectral measurements centered at different wavelengths by changing the temperature. The process that turned this insight into a viable measurement process follows.

This procedure is specific to the experimental apparatus and data drivers used and was built to quickly establish the proof of concept for the proposed solution. Better designs would obviously be used for the final system, which would increase reliability. Multiple iterations of each experiment were conducted. Frequently gross errors in the procedure would occur requiring more measurements, (e.g. misalignment of items or condensation on elements). This report describes the final setup; earlier experiments were conducted, producing inconsistent results that lead to process and/or apparatus changes. Across all designs, more than 300 measurements were taken, leading to the 50 consistent and repeatable measurements shown in the final experimental results.

The 3 main hardware components to the proposed solution are the photodiode, the IR-LED, the peltier cooler and the drivers for these items. Three IR-LEDs, 1 IR photodiode and a peltier cooler were purchased to show feasibility for the proposed solution. LED23a was used for the measurements and has a spectral response centered at approximately 2.3 μm, shown in FIG. 2. The rest of the IRLEDs were not necessary to demonstrate proof of concept, though basic experiments did show that while it is common for visible LEDs to be powered by a continuous voltage, the IR LEDs would overheat if powered continuously, so the manufacturer provided a controller that drives them with a pulse-width modulated signal. The driver effectively powers the IR-LED for some time, then power is removed and the cycle repeated. The maximum recommended duty cycle is 50% power at 16 kHz.

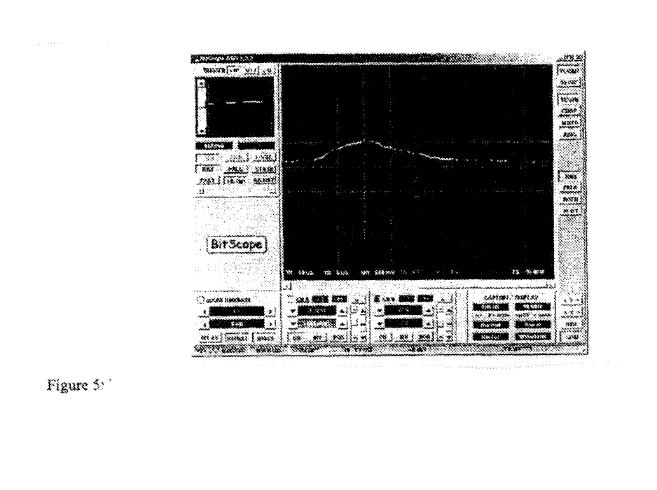

As shown in FIG. 4, a horizontal arrangement is being used, where the IR-LED (on bottom) transmits through a sample with the illumination falling on the IR photodiode at the top of the device. The output of the photodiode is measured and recorded using an oscilloscope. Because the LED is being driven in a cyclic pattern, the output of the photodiode measuring the light intensity is itself a wave pattern rather than a constant value. This varying signal in the photodiode complicates the measurement process and is discussed in more detail below.

The temperature of the IR-LED must be controlled and measured for the experimental process. The LED was thermally bonded to the peltier cooler and the driver was used to adjust the temperature. The larger distances and masses involved in this indirect temperature management and measurement increases the potential for error and also varies the speed at which the temperature changes and measurements occur. The temperature and photodiode output voltage measurements were time stamped and aligned in software. As described below, the measured voltage and temperature sequences were then processed and analyzed using Matlab.

In this the final experiments measurements of Colorado Springs tap water, a 95% ethanol mixture, a 40% ethanol/60% water mixture, a 150 mg/dl (an approximately 0.15% ethanol mixture) and a 15 mg/dl (an approximately 0.015% ethanol mixture) were taken. At least 12 iterations for each mixture were processed.

The mixtures were mounted on quartz slides, with a bead of hot glue around the edge of the slide to provide for a reservoir holding the liquid to be measured. Path lengths through the liquid were much less than 1 mm and difficult to control. The experimental process adjusts for total response so variations in path-length are less critical to the results, except that sufficient material is necessary to ensure the system does not saturate the photodiode at the lowest temperature, but does not have too much material so that there is still measurable voltage at the highest temperatures. The initial setup is done so that at the start temperature the voltage, as seen visually on the photodiode controller, is in a specified range.

The core steps to the data acquisition and processing of the procedure are:

Step 1: Record the data

Step 2: Sort the data

Step 3: Extract useful information

Step 4: Calculate Absorbance

Step 5: Calculate Mean Absorbance

Step 6: Calculate Mean Absorbance Derivatives

Step 7: Subtract Water

Step 8: Normalize Data

Step 9: Calculate Derivatives

Step 1: Record the Data

The output voltage of the photodiode driver was measured and recorded using an oscilloscope. Because the IR-LED is being driven as a cyclic pattern, the output voltage of the photodiode measuring the light intensity is itself a wave pattern rather than a constant value. The information from the signal is located in the region of the peak of the photodiode voltage. FIG. 5 shows the ideal voltage measurement needed for accurate ethanol detection.

Step 2: Sort Data, Smooth Over Time and Temperature

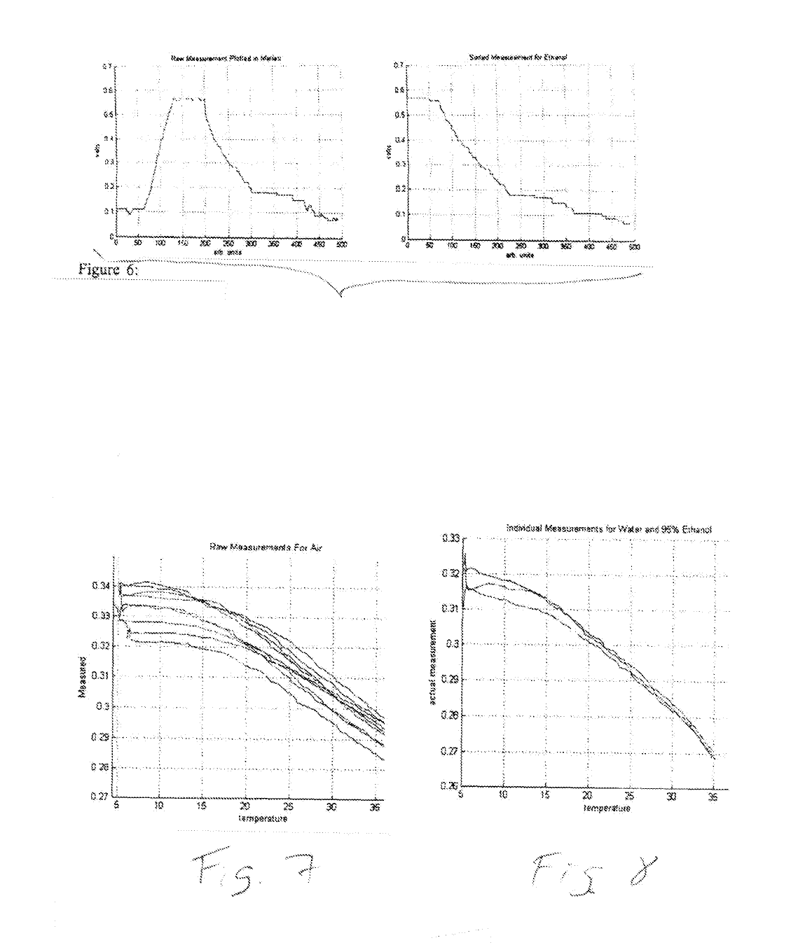

Next the data from the oscilloscope, such as that shown in FIG. 5, is saved and imported into Matlab for further analysis. A single measurement of intensity is then sorted from the highest voltage to the lowest voltage, making the measurements insensitive to the location of the actual pulse peak with respect to the scope's trigger for sampling. FIG. 6 shows this sorting. The top 250 values after sorting, approximating the value around the peak. The left graph in FIG. 6 shows the clamping or saturation effect toward the top of the pulse. This problem was initially overcome by offsetting the photodiode and IR-LED when the IR-LED was too bright, but this decreased repeatability. However, a similar result can be achieved by making adjustments in the current the IR-LED receives or in the amplification of the photodiode, both are adjusted.

Step 3: Extract Useful Information

Several problems were encountered with our simple approach to extracting useful information from the measurement. The peak of the voltage signal from the photodiode shifted between measurements and the oscilloscope, with 500 samples per cycle, measured much more frequently than the temperature sensor. This caused misalignments in the data. To address this, all measurements were time stamped, thus reducing the misalignment. There are 20-30 such single-pulse intensity estimations per temperature measurement. Each estimation of the spectral response of the medium is averaged per temperature measurement. This rate of sampling is sufficient to keep the SNR low enough.

FIGS. 7 and 8 show the raw data for multiple samples. Before further processing, the data is smoothed in time. This effectively smoothes over about ±2 degrees of temperature. As already discussed, the initial setup is adjusted based on the voltage reading of the photodiode controller to produce the same initial voltage. However, numerous variations occur between measurements which all contribute to the noise level. Several techniques to mitigate these effects were used, and many more have been designed.

Step 4: Calculate Absorbance

By employing an approximation of Beer-Lambert, absorbance is approximated from the smoothed intensity measurement (I) of ethanol, water or a mixture of water and ethanol, divided by the calibration phase smoothed average intensity of air (I₀) at the same temperature. This calculation gives the absorbance of the medium at each temperature recorded as:

A_{λ}=−log₁₀(I/I₀).

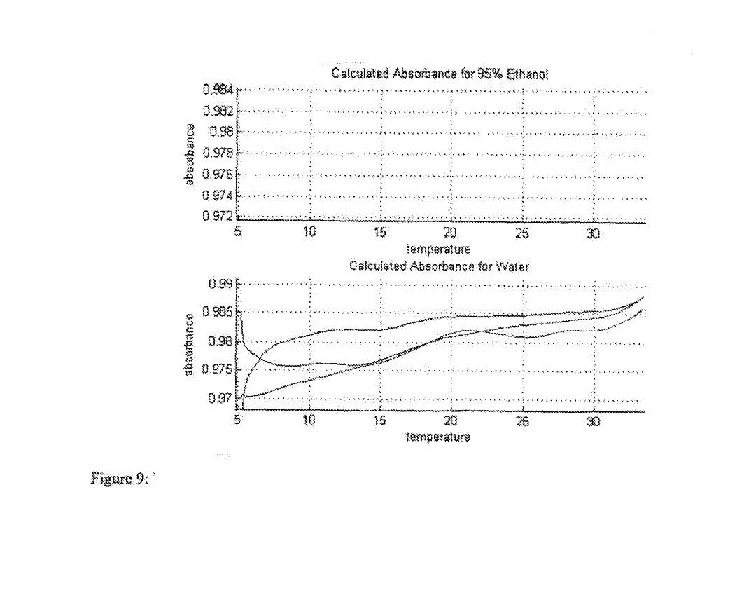

FIG. 9 show three runs of calculated absorbance for ethanol and water showing the range of variations seen. More runs were done but make the details hard to see in the graph.

Only one photodiode was being used in this experiment. This gave rise to more error because the normalization process uses measurements of air done at a different time and if there are variations in the LED output (e.g., because of current or temperature lag) these differences increase the noise in absorbance computation. This could be overcome by using beam splitters and multiple photodiodes, one for air and one for the medium, allowing the normalization to be on the actual IRLED output.

Step 5: Calculate Mean

The mean and standard deviations are then calculated from the smoothed absorbance calculations in FIG. 8, in this ease smoothing over 9 samples. These calculations clearly show the peaks and valleys of the spectral response in ethanol and water. The mixtures require some more analysis (Step 7) before the peaks and valleys of their spectral responses can clearly be seen.

The ethanol curve shows dear peaks and valleys and the standard deviations are small enough to see that the sizes of the peaks and valleys are well within our measurement tolerance. While water shows some inflections, there are no peaks/valleys in the region of interest.

Step 6: Calculate Derivatives

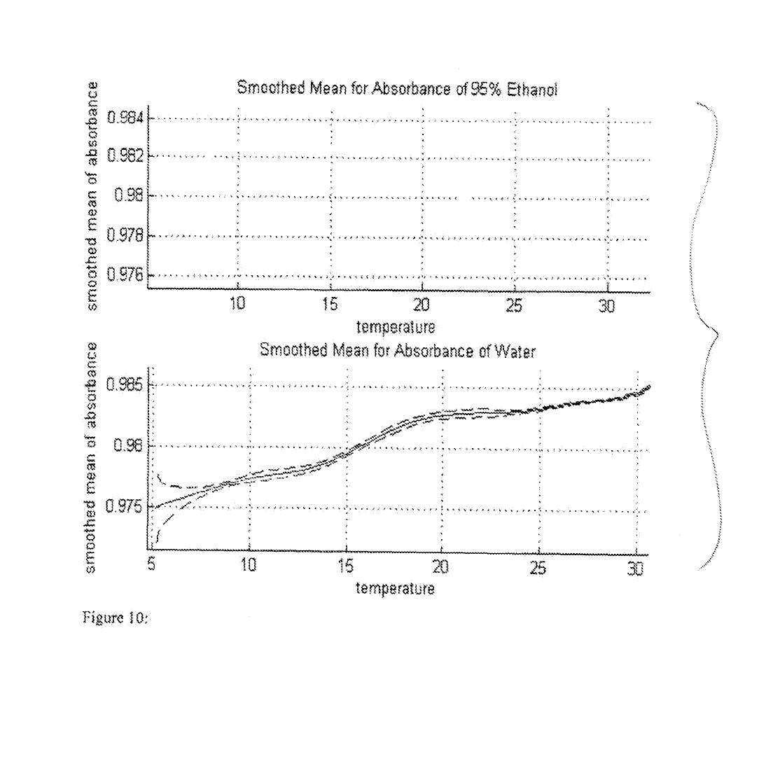

The derivatives are then calculated using the values obtained from the smooth mean from Step 5 (FIG. 10). The derivative is computed for each curve as well as the mean derivative and its standard deviations (shown as dashed lines). The derivatives show the peaks and valleys of ethanol at zero crossings. The scale is 10-4 in part because of the fine sampling in time/temperature, as the LED is pulsing at 16 KHz, so the horizontal sample between pulses is very small. As expected from the mean plots, there are visible zero crossings for the ethanol, but not for the water. However, the fact that the standard deviation boundary of water does cross zero in regions where zero-crossings are expected for ethanol, does suggest care will be needed to drive the standard deviation lower in the actual system.

Step 7 and Step 8: Dealing with Mixtures: Subtract Water and Normalize

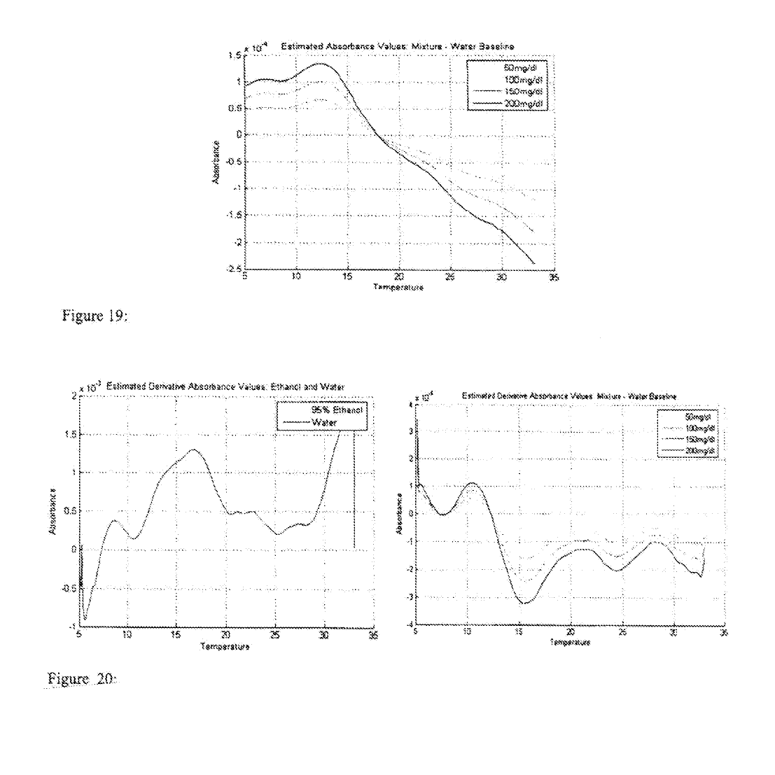

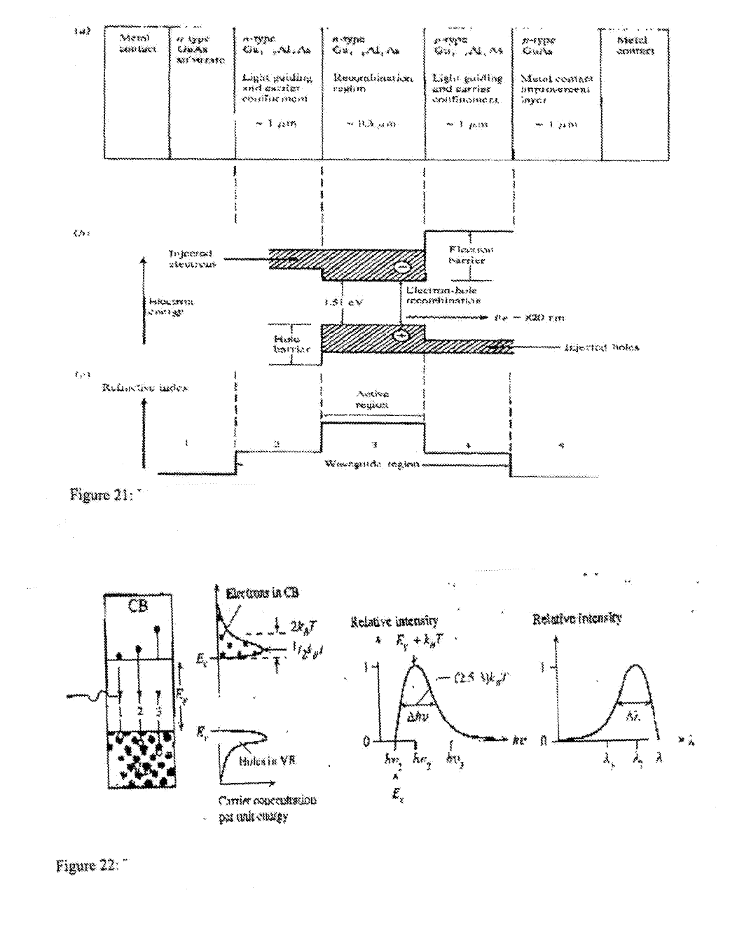

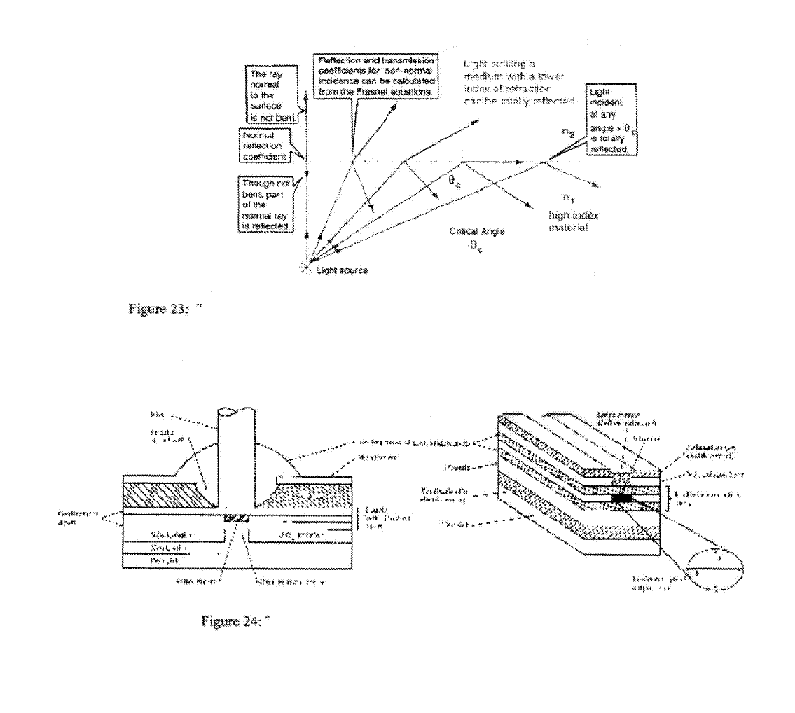

When combining mixtures of material the resulting absorbance will be a combination of the underlying materials. For low ethanol mixtures, there will be no visible peaks and valleys because increasing proportion of water will dominate the overall absorbance. Normalizing, by subtracting out the response of water from the measurements, addresses this issue. (For a final system this would probably involve subtracting out skin response, and it will be important to determine if it must be person specific). This is followed by a normalization process, mapping the resulting data to [0, 1]. The 40/60 ethanol mixture still looks a lot like ethanol and has very clear peaks/valley of ethanol, though somewhat shifted. The 0.015% and 0.15% have similar shapes, but are still moderately different from ethanol, possibly from differences in measurements (or LED output) during the measurement of water (which is subtracted). Rather than just subtracting water, a better approach may be to model the overall data as: y=x(CH₃CH₂OH), solving for y in the least squares sense over the database. Still these results are promising even in concentrations as small as 15 mg/dl (shown in bottom left) and consistent with the results from simulations: see FIGS. 19 and 20.

Step 9: Calculate Derivatives on Mixtures

By taking the derivative of the normalized absorbance the peaks and valleys become more apparent. The derivatives of the normalized data show how the peaks and valleys of ethanol stand out when the absorbance of water is subtracted from the measurement for the mixtures in 15 mg/dl and 150 mg/dl. The derivatives for the normalized data change the scale as a result of the normalization that occurs. This is why the raw derivative for ethanol is smaller than those for the normalized mixtures.

These results are promising but not without some errors, as there are two added zero crossings and some shifting of the zero with respect to ethanol. For idealized data, with subtraction of water, the shifting should be smaller and there should not be significant added zero crossings. Errors in temperature measurement, relative to the actual LED temperature, would cause localized shifts and produce the increasing slope of water, and decreasing slope of ethanol, could introduce the spurious zeros.

Specific Difficulties in Measurements

Along the path to the above experimental analysis, hundreds of experimental trials were attempted sometimes showing inconsistent measurements. As the experimental process was refined the following issues significantly impacted measurements and experimental repeatability. Some of these were oversights, e.g., the IR absorption of the Petri dishes and beakers, and easily removed once identified. Others were more problematic.

Condensation and Lens Distortion

Condensation and lens distortion were two factors played the most significant roles with regard to the problems encountered with repeatability and incorrect readings. When the LED is made colder than the dew-point condensation, and sometimes ice crystals, would form on the lens. Light would pass through the water or ice crystals and cause erroneous intensity measurements. One possible way to avoid this would be to use a LED with the response in higher temperatures, thus discouraging condensation. Others would include having the TEC inside a sealed dry environment. The experimental setup was improved by adding an AC unit to the darkroom and reducing the ambient temperature and humidity to reduce the potential for condensation.

Alignment and Intensity

Alignment and intensity is critical for successful measurement of ethanol. The photodiode amplifier may over-saturate the signal if the intensity is too high. If oversaturation of the amplifier occurs, the changes in the temperature sweep will still occur but the change in intensity will be lost. In particular air requires lower intensity than water and ethanol, and variations in the output/measurement caused by differential driving increase the experimental error. The initial experimental setup used alignment of the photodiode to adjust the intensity value. During this process a slight movement in the alignment of the photodiode would change the measurement drastically and hinder repeatability. However, once proper alignment and intensity was achieved (as long as the experimental setup was not modified) the results were repeatable. The values displayed on the photodiode driver as the temperature increased correlated to the values as the temperature decreased.

Temperature Control and Temperature Measurement

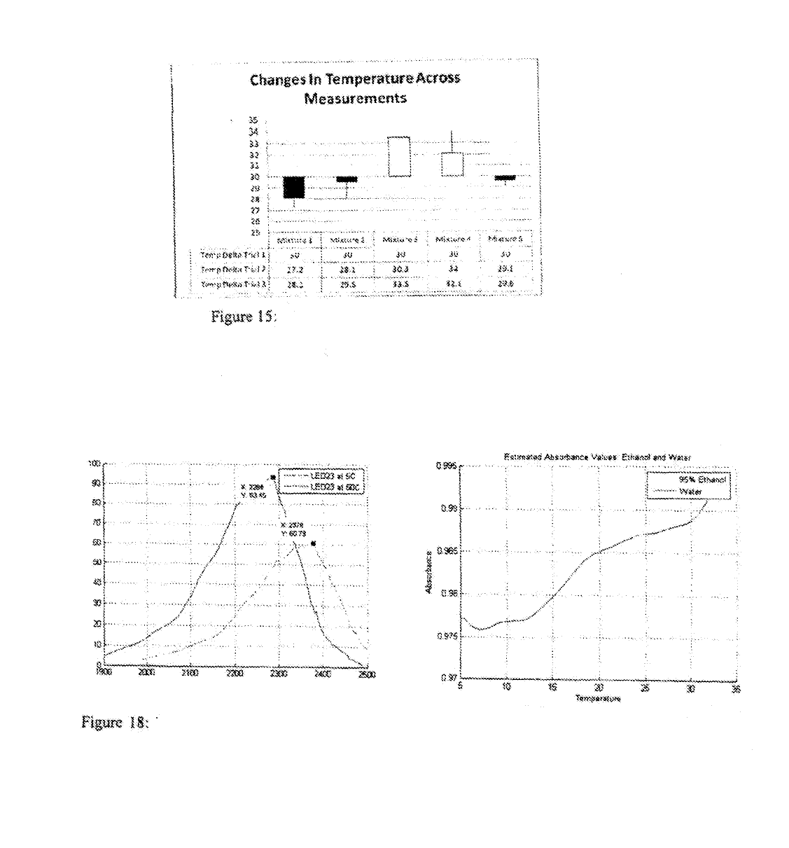

The temperature measurement and control for this proof of concept is relatively weak and an area where there can be substantial improvement. Because of the high level of dependence on temperature, measuring the temperature change accurately is critical. The most significant issue for our measurements was the fact that the temperature sensor was measuring the temperature on peltier and was on the outside of the LED case, rather than on the LED itself. Thus, the temperature of the LED could be different than the change in the external temperature, and the difference could be temporally varying causing more error. FIG. 15 looks at the data a different way and shows the differences in measured temperature for the same measurement. The change of intensity for the LED is the true indication of its temperature. The actual temperature measurement was varying by as much as 3 degrees and possibly more. For a final system, the TEC could easily be integrated in the LED package, and a coupled TEC-LED should be used.

A secondary advantage of directly cooling the LED is that cooling it directly would reduce the thermal mass that needs to be adjusted, increasing the speed at which the temperature sweeps could be conducted. Another issue is that our current temperature sensor sampled at 1 Hz and frequently dropped packets causing more error in the measurement. The sensor measures temperature at 0.1 C resolution, which may be sufficient to show difference in peaks and valleys for ethanol, but limits alignment in the case of subtractive normalization needed for mixtures.

Experimental Conclusions

The experimental process in research is often a meandering process, full of unexpected obstacles to be overcome. This experimental development had many such side turns, but in the end developed a protocol that shows strong promise for alcohol measurements in the ranges needed. Repeatable measurements that detect peaks/valleys from alcohol at 0.015% were obtained using TMS with a rudimentary apparatus built with plywood frame, quarts slides and plenty of hot glue. Many engineering challenges remain to prove it in actual subjects and design a better device/process to reduce errors, but these experiments show basic feasibility of the concept.

Simulation modeling and results will now be discussed.

There are two parts to the simulation. The first part was completely theoretical, and preceded all experiments, the second part was to relate back the experimental data and allow more what-if based on the theory.

All of the spectral responses are interpolated from manufacture datasheets. In the first part of the simulation GaSb substrates are assumed and it was further assumed that their spectral responses are temperature dependent: As temperature increases their peak value shifts and their efficiency decreases.

This change was modeled as a linear shift and scale. The shift and scale for an emitter with a peak located at 2350 nm is shown in FIG. 2. This information can be used to interpolate where the expected values of peaks and valleys occur at a given temperature. The spectral response in between the LED spectral peaks at the two measured temperatures will be the where the response of the medium appears. This means there is a non-linear relationship between the temperature change and the wavelength of a substance.

The simulated method is a voltage sweep from 5 C to 50 C. As the temperature changes the emitter's response becomes more or less efficient depending on a decrease or increase of temperature. This change causes the spectral response to move left or right, sweeping across a portion of the medium's spectral response. The change in the emitter can be divided out, using the Beer-Lambert law, leaving the response in transmittance or absorbance of peaks and valleys in a given medium. This process is simulated with data from two actual emitters: LED 23 and LED 22 and modified data from a hypothetical emitter: LED 22—modified. The targeted peaks and valleys of ethanol in the region of interest do not have a COTS IR-LED available giving the shift needed. Feasibility for this region was shown by gene rating a specification for a desired emitter and then simulated. By using the spectral data on the datasheet a curve was interpolated. This curve is then shifted right and scaled down slightly as the temperature is increased.

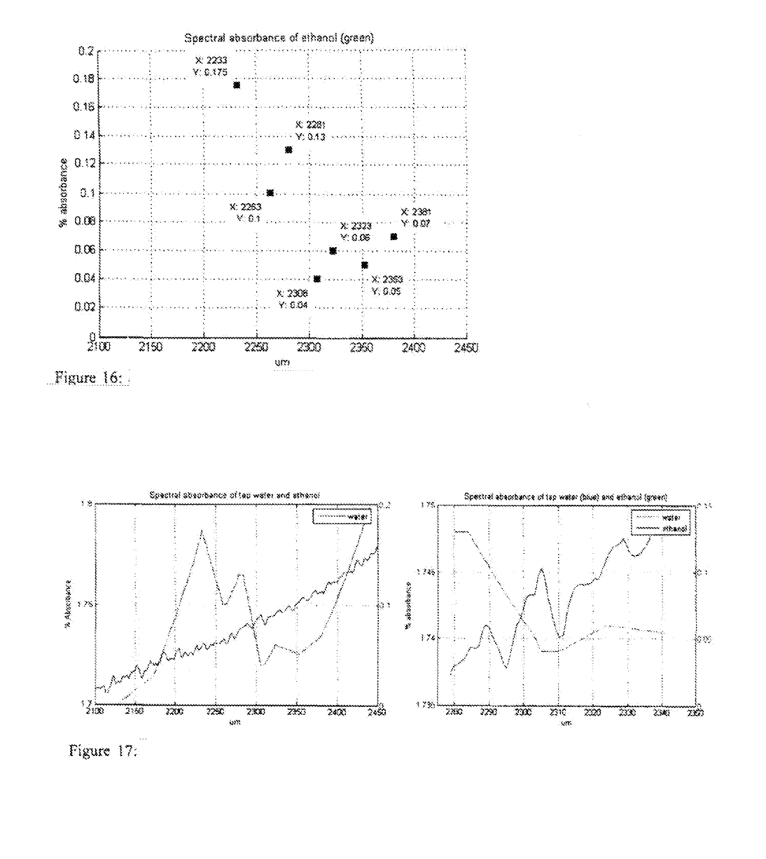

The theoretical spectral response of ethanol is shown in FIG. 16, with the key peaks and valleys indicated. The spectral response of the LED 23 at 14 C and 24 C and 53 C is shown in FIG. 2. There is a clear separation of energy levels, but there is a less distinctive shape in the response curve at the higher temperatures. The LED emissions (ignoring alt the pulses with issues) were simulated and adjusted for the materials absorbance of light. We then computed the photodetector response (from the manufacture's spec sheets) to produce the expected response at a particular temperature.

The second part of the simulation combined actual data for pure ethanol and water with simulated mixtures of the two. This provided a quantifiable measure to determine if actual tests are consistent. Once the baseline measurements for 95% ethanol and water were established theoretical measurements of several different mixtures were made. This data showed that water must be subtracted in order to see the effects ethanol was having on the mixture. While water made the most sense to be used in this ease any base-line spectral measurement, such as human skin, could be used. The graphically displayed results are shown below.

Simulation Results: Part 1

The first part of the simulation provided intuition to the region being scanned and helped us determine if the process was feasible and worth doing physical experiments. The initial work used models which were somewhat inaccurate but still showed the potential of the TMS approach, and justified using TMS in the actual experiments.

Simulation Results: Part 2 Relating Back to Real Data

The second state of simulation was feeding back data from the real measurements to help analyze SNR and determine if a viable approach existed. This helped develop the algorithms and address ideas on how to deal with calibration. The simulation graphs are slightly different from the actual data, e.g. the water measured had more slowly increasing absorption, but in general, results were consistent with the actual data.

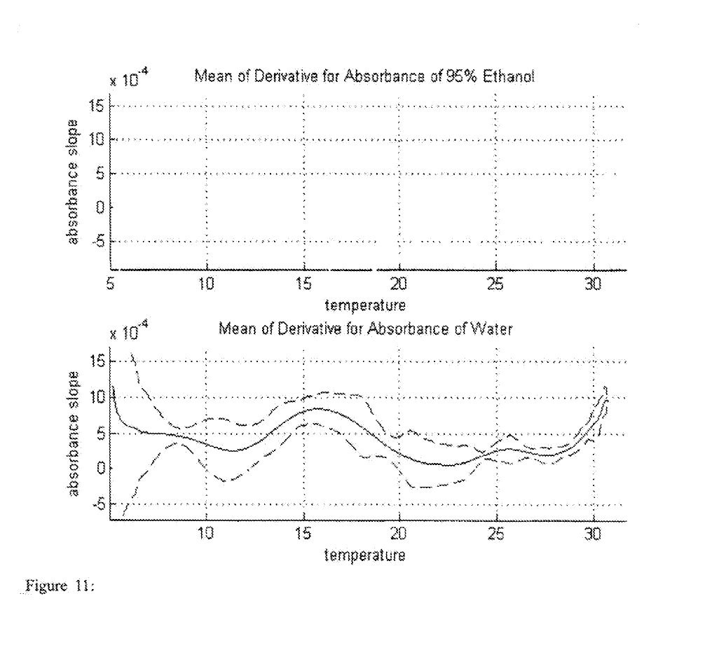

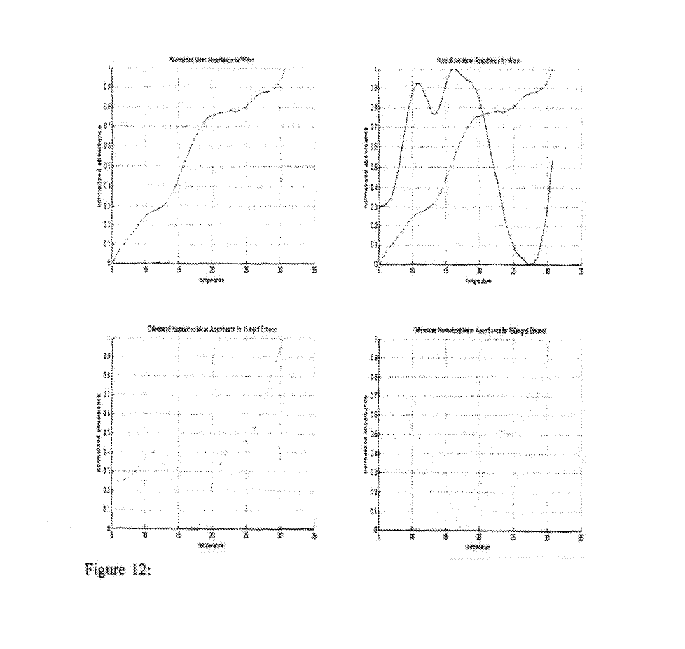

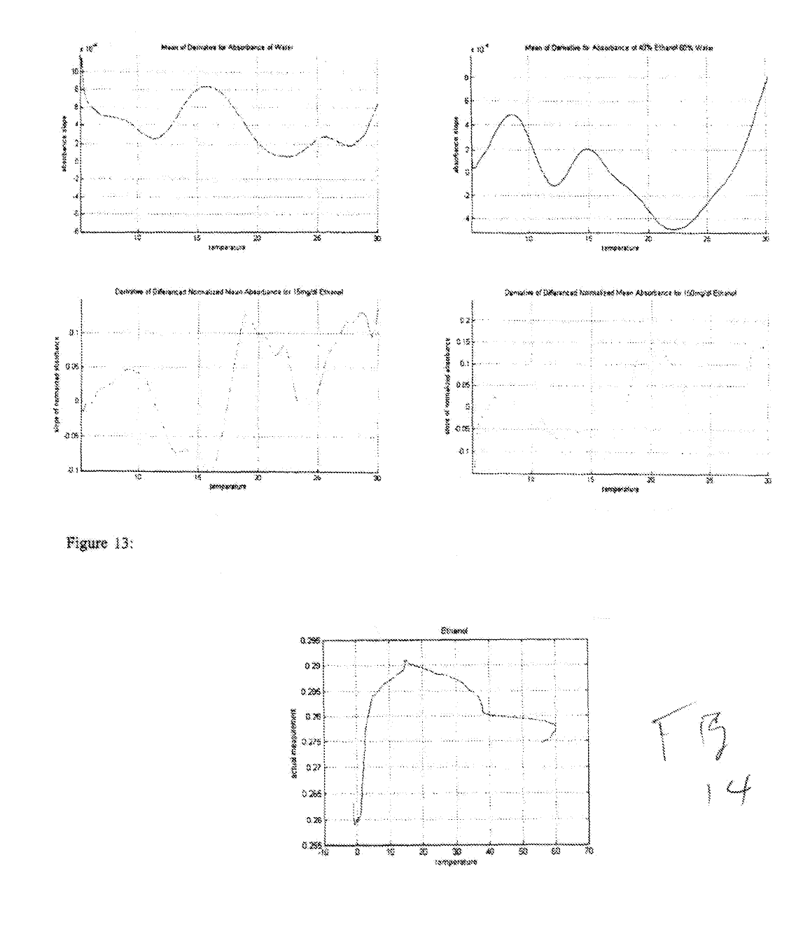

This part of the simulation now follows Steps 5 and 7 in the actual procedure: Subtract the absorbance response of water from the measured absorbance of a mixture and find the derivative of the differenced value. The simulation suggests the location of the peaks and valleys of the mixtures should be closer to the pure ethanol than our measured value, but overall there is good agreement. In particular, between 15 C and 20 C. FIG. 19 show all mixtures have values that cross 0, due to the intersection of water and ethanol shown in FIG. 18 (right). This is consistent with the actual measurements shown in FIG. 10. Comparing FIG. 10 to FIG. 12, it can be shown that the simulated response is reasonably good match to the data for the lower temperatures, but while the real data shoots back up to larger absorbances at higher temperatures, the simulated data continues downward. This is probably caused by whatever resulted in our water measurements not increasing as much as the simulated water response. (Possible causes are variations in water content, e.g. chlorine or locally dissolved minerals or it may be caused by the normalization with air being inconsistent.). Similarly, our derivatives for the lower temperatures are reasonably well matched in simulation (FIG. 20) and actual (FIG. 13), but at higher temperatures there is a noticeable departure.

Background on the LED will be provided.

An ideal LED should have a high radiance (light intensity), fast response time and high quantum efficiency. These characteristics are best achieved via double hetero-structure devices. A double heterostructure semiconductor device has junctions between different band-gap materials. It is important that the region in which recombination occurs there is a high carrier concentration. The double heterostructure enables the source radiation to be much better defined, but further, the optical power generated per unit volume is much greater as well. When the structure is connected the Fermi level must remain constant at thermal equilibrium. Because the middle p-layer is smaller in band gap than the other two layers, when the structure is forward biased electrons would flow to the middle p region but would be confined in that region, since there is a potential barrier due to the difference in band gap, limiting them from diffusing further in the adjacent p region. When the electron combines with a hole from the other side of the gap a photon is created. The energy of the photon is a function of the separation energy between the electron and the hole.

By keeping the middle layer extremely small (−0.1 μm) the emitted photon can be confined to a very small area and photons generated in other layers cannot be absorbed since it will have a different energy value than the band gap of the middle layer.

Emitted wavelength depends on band-gap energy. The order of increasing voltages is the order of increasing energy required for emitting light from the LED. The wavelength of light emitted depends on the band gap energy, depending on how strongly the bonding electrons are held in localized, depending on the size of the atom, some small atoms hold their electrons more tightly.

Temperature Dependence

Temperature dependence of the LED is critical to our TMS concept. As the temperature increases the diode gain decreases and more current is required to overcome the losses and produce forward bias. By increasing the temperature, the energy of the electrons and holes increases allowing more to be free outside the active layer (in the n and p layers). More recombination happens outside the active layer with free carriers that would have reached the active layer but recombine instead. FIG. 22 shows the distribution of electron-hole pairs and the function of energy and temperature.

Higher energy band-gap means longer wavelength:

λd(FinalJunctionTemperature)=λd(InitialJunctionTemperature)+ΔT*γ(nm/° C.)

In other words for every change in junction temperature there is a change of W nm in dominant wavelength λd, where γ is a function of materials used and geometry of device. Usually this is considered a problem to be controlled, but in our case it allows us to sweep the wavelengths of the LED output over a range.

Reflection/Refraction is temperature dependent as it changes the effective index of refraction of the material compared to air. The impact is greater at larger angles of incidence and it shifts the angle at which TIR occurs. This impacts both optical spreading of light emitted and spectral spreading/shifting of light emitted. This is a much smaller effect, mostly impacting the wavelength spread, allowing edge-emitting LEDs to maintain a narrower beam with reduced wavelength spreading.

The radiation emerges in the direction perpendicular to the junction plane for SLED's, so SLEDs emit light over a wide area giving a wide far-field angle. SLEDs are more commonly used in communication as they support a more efficient coupling to the optical fiber than edge emitting LEDs. A SLED has an active region (the part of the LED actually emitting photons) from 20 μm to 50 μm.

This is in contrast to the ELED. The primary active region of an ELED is a narrow stripe, which lies below the semiconductor substrate. The substrate is then cut or polished so that the stripe is runs between the front and back of the device. These polished surfaces are called facets. The rear facet is highly reflective and the front facet is antireflection-coated. The rear facet reflects the light so it all travels out the front of the LED. ELEDs emit light in a narrow emission angle resulting in a narrower spectral line width and are typically more sensitive to temperature fluctuations than SLEDs.

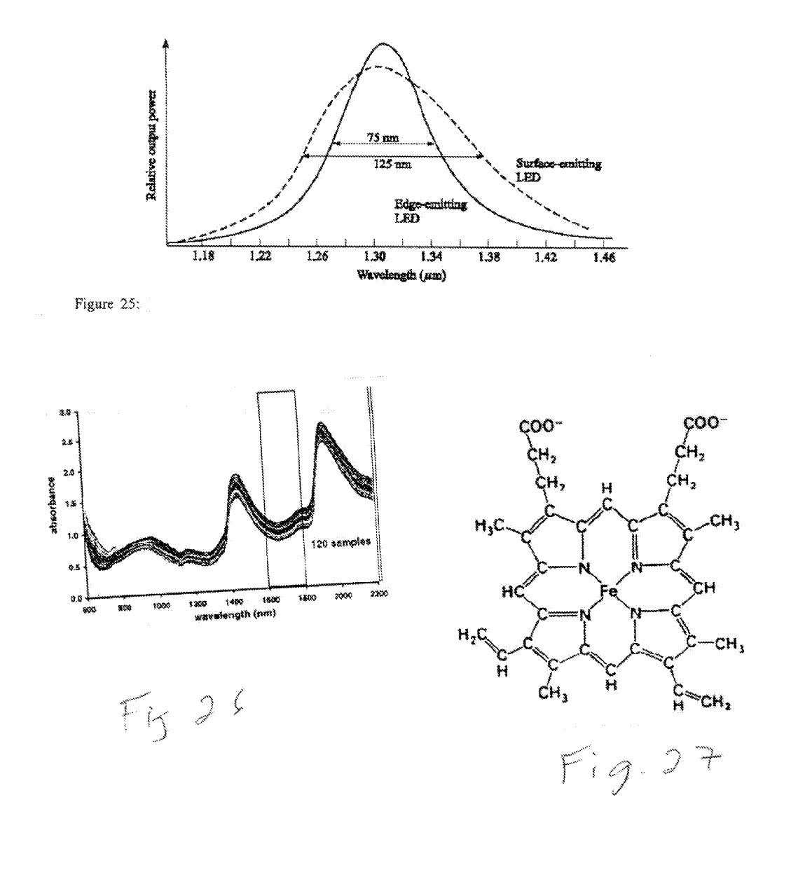

Because the TMS approach is sweeping the pulse across the sample, a narrow sample beam would help and so if possible, an ELED would be preferred. FIG. 25 shows some example variations in spectral spread for NIR LEDS. The spread of the IR LEDs uses for our experiments is even larger, which is predicted by the theory.

A part of the effort was to assess the measurement feasibility of ethanol (CH₃CH₂OH) at various skin sites to determine the blood alcohol concentration of a subject (SAC). The spectral response region of ethanol to be investigated is in the 2.2-2.4 μm and possibly 1.6-1.8 μm, both areas are highlighted in FIG. 26. Some recommendations will be made regarding where the sensor(s) needs to be placed on the body to get data of sufficient quality so the measurement of ethanol can be meaningful for commercial deployment. This includes examining other work and critical factors of the skin that are going to impact the measurement.

Aside from plasma, which contains about 90% water, red blood cells are the most predominant structure found in blood. The oxygen carrying protein in the red blood cells is called hemoglobin. The chemical formula for hemoglobin is C₃₄H₃₂FeN₄O₄. The chemical structure of hemoglobin is shown in FIG. 27. The double C bonds and C—H bonds produce features are in the spectral regions of interest. This will need to be accounted for in the measurements.

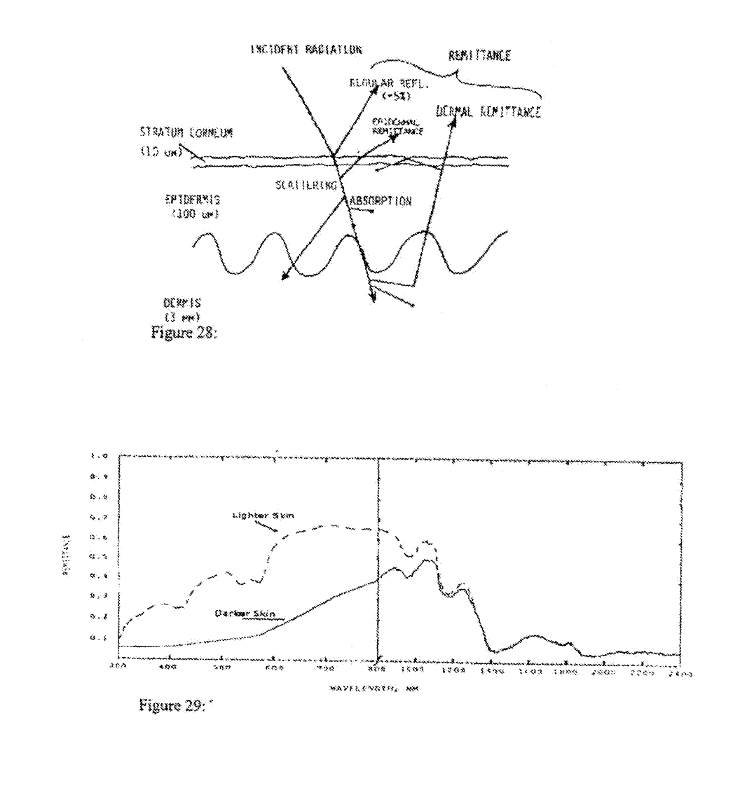

Regardless of the site at which ethanol levels are measured, light will have to come in contact with skin. Along with differences in concentration of melanin various skin conditions need to be examined as well as the changes of the spectral absorptivity of skin due to temperature changes. Ideally the ethanol measurement device will function uniformly throughout the range of skin colors and conditions. While many studies exist on the spectral properties of human tissue, few exist which discuss the absorption or transmission of skin at wavelengths longer than 2.4 μm. A study was done on the differing spectral characteristics of skin color. FIG. 28 shows the behavior of light as it passes through the skin. A small fraction of an incident radiation is reflected due to the change in medium at nearly perpendicular applications. This will need to be accounted for in the measurements as well as the rest of the remittance (diffuse reflectance) though the epidermis and dermis. Because skin is not smooth, there will always be some sort of remittance of light: the more dry and flaky skin is, the more light may tend to scatter more. For normally incident radiation, the regular reflectance of an incident beam from normal skin is always between 4% and 7% over the entire spectrum, from 250 nm to 3000 nm for dark and light skin. However, when determining the penetration of optical radiation within the dermis, the scattering by collagen fibers is critical.

The next factor to be considered when light enters the skin is the scattering of light. The spatial distribution and intensity of scatter light depends upon the size and shape of the inhomogeneities relative to the wavelength, and upon the difference in refractive index between the medium and the inhomogeneities. By having small variation of tissue present in the measurement sample, less scattering can be expected, producing a more accurate measurement of ethanol. This guides the measurement to an area with relatively little muscle or other interfering tissue, namely the fingers and upper palm. The Kubelka-Munk theory can offer a simple quantitative treatment of the optics of the skin.

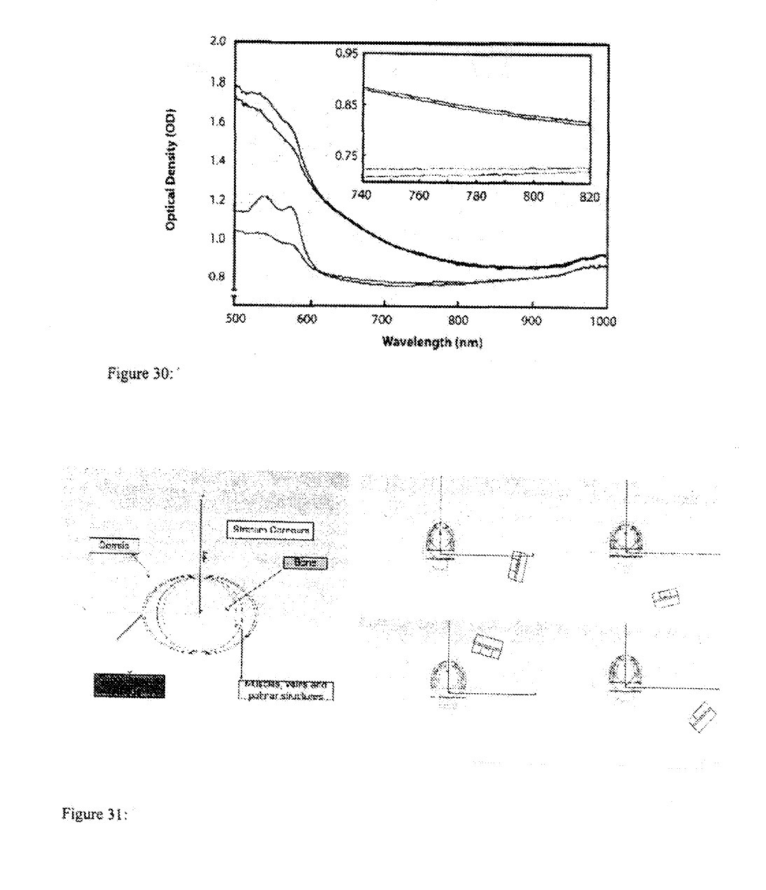

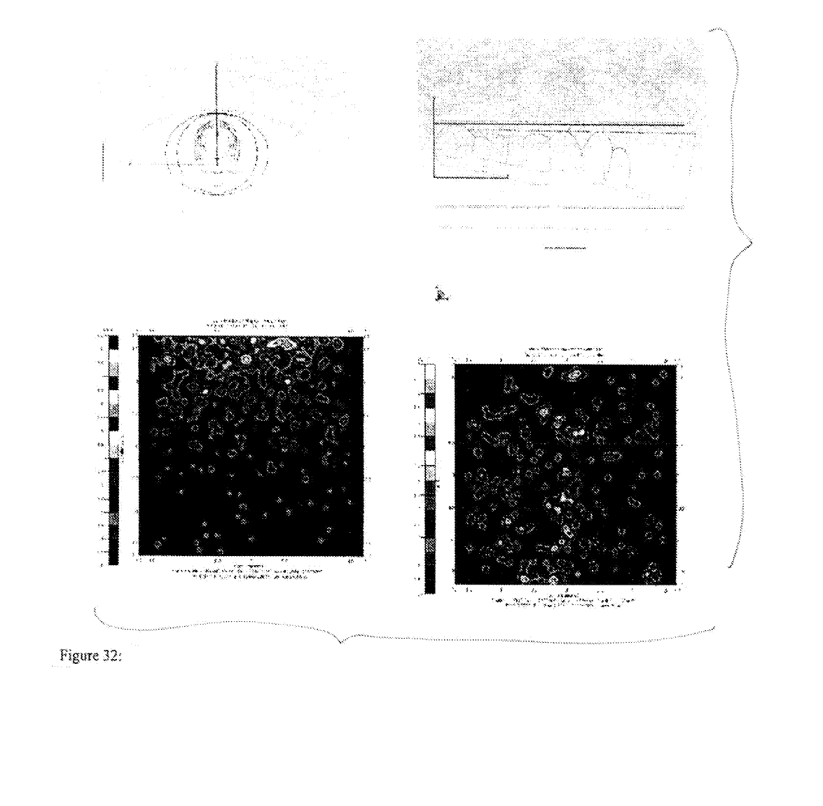

FIG. 30 gives the remittance of dark skin and lighter colored skin. The graph does cover the 1600-1900 angstrom range and some of the 2200-2400 range but not all of the wavelengths examined, but it does show a trend in both similar levels of absorption and the lack of absorption at wavelengths over 1.1 μm. This is of great benefit measuring ethanol at longer wavelengths because at these wavelengths the skin's effect on measurement, regardless of pigmentation, is negligible, only accounting for 5-7% of the total transmitted light. The absorption spectra show no significant change with temperature in any direction. This study was conducted with various layers of skin with several varieties of skin color and tissue from 0.65 μm to 1 μm, but the trend showed the absorption to decrease at longer wavelengths. No work was found on subcutaneous tissue in the wavelengths of interest. However, absorption spectra show the expected features due to tissue chromophores, and the transport scattering coefficient exhibits a steady decrease with increasing wavelength. This lends evidence to believe that these areas will have little effect in the region of interest or the effects which can be accounted for.

Some nonmelanoma skin cancers have shown statistically different scattering at the wavelengths between 1.05 μm to 1.4 μm. Another study on the blanching of skin also found that the spectral response of skin seemed to converge at the longer wavelengths. Some of these results are shown in FIG. 29. While light and dark skin may differ, the response of ethanol at the longer wavelengths is large enough, would be noticeable regardless of the color of skin. No known work could be found which was conducted for skin conditions in the longer wavelengths, so it cannot be concluded if nonmelanoma type skin cancer or skin blanching or other skin conditions would change scattering or absorption results at these wavelengths. However, the Department of Forensic Science, Virginia notes that “The chances of another compound having the same IR pattern [as ethanol] in the 3 μm-4 μm are remote.”

Multiple types of sensors can be used, including:
DIDS: discrete narrow band illuminates with discrete broad spectrum sensors, very likely using multiple illumination sources and multiple sensors with different spectral response characteristics.
MIDS: broad spectrum illuminates with dispersion into a device that masks various wavelengths light, passing the results through the tissue and then imaging them onto discrete sensors. The mask may include movable components/slits or mirrors.
EIDS: broad spectrum illuminates with dispersion into a device that encodes temporally varying combinations of light, which pass through the tissue and that are then imaged onto a discrete sensor. The encoder may include a Digital Mirror Device, an acoustic-optical crystal or maybe a rotating medium (though vibrations might be an issue).
DICS: discrete narrow band illuminates, with dispersion to an array sensor where the array sensor is something other than a traditional (and expensive) InGaS sensor, e.g. a specially coated CMOS sensor. A spatial varying coating will be explored to allow increases sensitivity, building off the ideas in Dr. Boult's INSPECT approach.
CICS: Continuous illuminates with dispersion to an array sensor where the array sensor is something other than a traditional (and expensive) InGaS sensor.

Examining the above list it should be clear that all of these except DIDC, and possibly DICS, require a dispersive element to spread the wavelengths for measurement. Thus looking at approaches for low-cost dispersive elements was the first element we considered. The dispersion or diffraction is only controllable if the light is collimated, that is if all the rays of light are parallel, or practically so. A source, like the sun, which is very far away, provides collimated light. In a practical monochromator however, the light source is close by, and an optical system in the monochromator converts the diverging light of the source to collimated light. There are two major choices for the monochromatic in dispersive elements, ones that use focusing gratings that do not need separate collimator, and the more common (and cheaper) use collimating mirrors. Reflective optics are preferred because they do not introduce dispersive effects of their own. The most common approach is the use of a Czerny-Turner monochromator, which greatly reduce the total size at only a modest increase in cost.

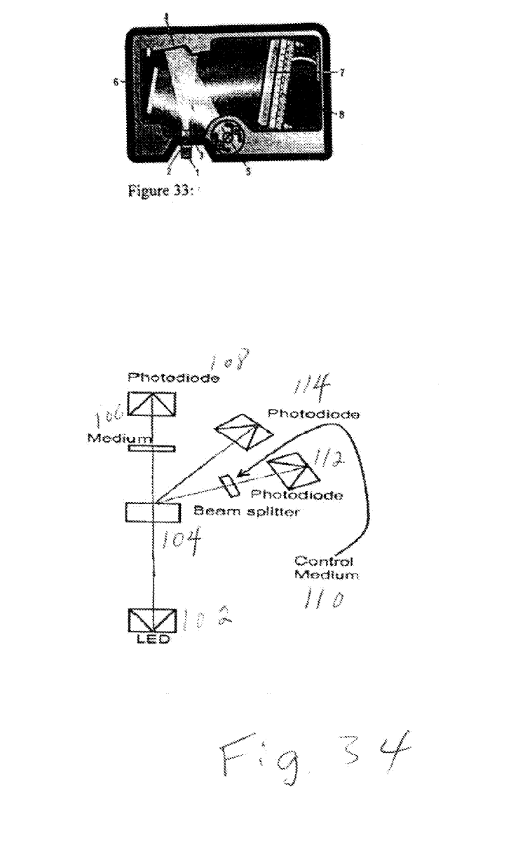

To deliver light to a sensor, we analyzed one of the most widely used compact dispersive elements, the classical asymmetrical Czerny-Turner optical bench. One of the possible configurations is shown in FIG. 33. The system will have no moving parts that can ware or brake (other than possibly the sensor element if it is a discrete sensor). All the other optical components would be fixed in place at the time of manufacturing. Light enters through (1) and passes through the installed slit (2) which acts as the entrance aperture. We will consider the rectangular shape of the slit with the high of about 1 mm and width in the range of 5-200 μm. The slit size will determine the amount of light entering the bench and, indirectly, the final pixel resolution of the CMOS detector. Next, light will pass through an absorbing filter (3), and then reflect from a collimating mirror (4) toward a diffraction grating (5). (For testing the grating could be installed on a rotational platform to select the starting waveform we will specify. After final selection, one could permanently fix the chosen position in place to eliminate mechanical shifts or drifts. The final construction costs would be lower than in intermediate system, but still requires very high alignment criterion so manufacturing would still be modest). Finally, the focusing mirror (6) will focus the first order spectra on a collection lens (7) and a CMOS or discrete detector or wavelength selector DMD. The cylindrical lens(s) may be needed to focus the light from a tall slit onto the shorter detector elements to increase the light collection efficiency. Building from results in the literature and related products on the market, the minimum size of this type of system is 4″ for a system with F4 (focus point 4×), and it could be smaller (2″) with F4 (focus point 4×), which would require more light and a much more sensitive receiving element. The lowest cost estimate for the grating, optical elements and precision assembly and was well over $200, before considering the sensor or processing, making anything needing such an element outside the cost parameters.

The emerging use of nanowires for narrow band-pass optical filters became another topic of interest. The basic concept is that a very fine array of nanowires produces a localized surface plasma resonance. The resonance produces a very narrow band-pass filter, often with half-widths around 50 m and peak-to-peak separations of 10 nm in the visible range. Early nanowires were done for polarization techniques, but required Electron Beam Lithography making them expensive. More recently, the process of production of nanowires has been demonstrated at the University of Pittsburgh, using a much lower cost imprinting technique; potentially allow direct application on sensor chips. They have licensed the technology and prototypes for the visible spectrum are now in production by Nanolambda, though yields on the imprinting process are still pretty low. Similar to a Bayer pattern, with this concept placement of a wide range of narrowband filters directly on the CMOS sensor can give a similar effect. The concept extends down to the 2000-3000 nm range, thought the half-widths get broader because of the constraints on the nanowires needed to sustain the surface plasma field. This approach has not been demonstrated physically in the wavelength of interest, but it should apply.

One concern for the nanowire approach is the cost of the imaging array. The imaging array may be why HP never pushed once they looked into it. While CMOS imagers are particularly cheap, the material and lower volume in the spectral range needed for our application will make the nanofilter+area sensing approach more expensive.

A mixed approach using a collection of the nano-filters combined with the TMS approach with a small number of photo-detectors is also a possible area for feasibility. This approach is different from the standard filter usage for broader spectrum measurements which involve spatially separating the measurements of incoming light. This approach may increase the efficacy of TMS by sweeping in temperature and time and so modulating the sensor response or LED output around a few regions of interest then effectively measuring the sum of them. The concern of light levels is still an issue, as these filters will reject most of the light impinging on them, so covering the photodetector with an array of these filters would significantly reduce overall efficiency of the sensor. An unexplored solution may be placing the filters on the cover glass of the LED, which may be more effective as the rejected photons can be reflected down ward back into the reflector surrounding the LED and eventually have a chance of emitting through the small part of the filter that is appropriate for it.

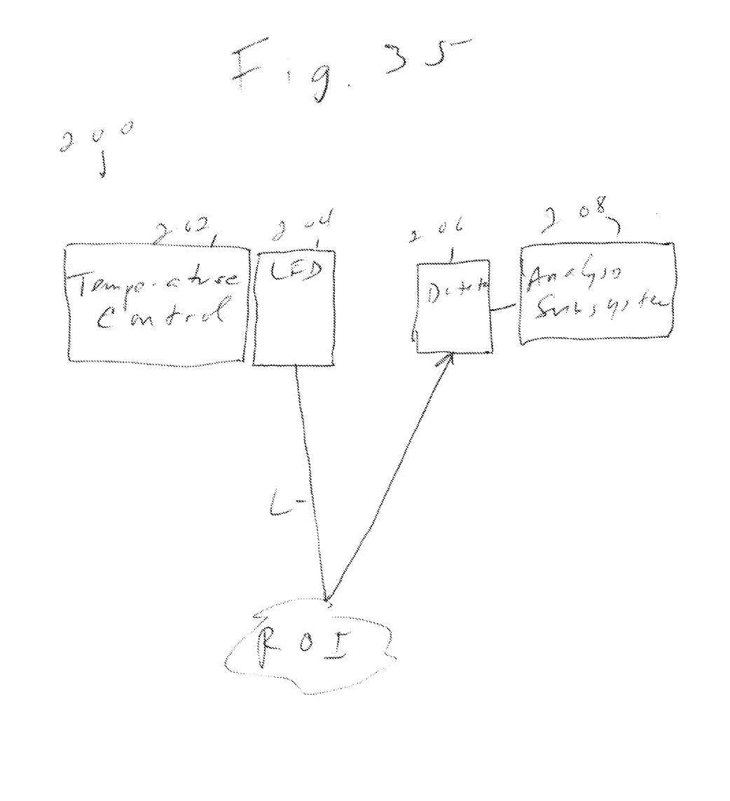

The most significant errors are most likely caused by the variation in responses and need to calibrate to absorption (dividing by the response in air) and dividing by water since both of these are using measurements from the LED at a different point in time. A beam-splitting approach with multiple photodiodes would increase costs but have the potential to simplify these issues. FIG. 34 shows two different models for beam splitting that can be used to calibrate for air and/or a known sample. As shown, light from an LED 102 passes through a beam splitter 104 on its way to the medium 106 and a photodiode 108. The beam splitter 104 splits off a portion of the light for normalization. In one alternative, the portion of the light passes through a control medium 110 to a photodiode 112. In the other, the control medium is omitted, and the light passes directly to a photodiode 114. The beam splitting can balance the expected light levels and simultaneously address any problems of differing LED output variation over time.

The second potential major source of error is the limited temperature control and measurement. A diode with a built-in peltier cooler (see above) would help this situation, as would a more controlled test environment. Cooling the diode using gas also seems to have a residual effect on warm-up rate.

The third major source of error is the variations in physical measurements caused by the limited experimental setup. A vertical or sealed testing apparatus would solve that problem, as the path length would be the same between measurements. Reflective measurements would also solve the problem.

There are multiple issues in the current setup that can be corrected for liquid measurements and others for reflective measurements.

In summation, FIG. 35 shows a system 200 embodying the preferred or another embodiment. A temperature-control subsystem 202 controls the temperature of an LED 204 such that the light L emitted by the LED sweeps across a range of wavelengths. The light L is reflected from (or transmitted through) a region of interest ROI, from which it is made incident on a photodetector 206. The photodetector 206 outputs a detection signal to a spectroscopic analysis subsystem 208, which can be any suitably programmed computing device.

The spectroscopic analysis subsystem 208 analyzes the detection signal to detect the peaks and valleys corresponding to the known spectroscopic peaks and valleys of the analyte, e.g., ethanol. By detecting and measuring the peaks and valleys, the spectroscopic analysis subsystem can determine both the presence and the concentration of the analyte. In the example of ethanol, the spectroscopic analysis subsystem can determine the presence and concentration of the ethanol and use that information to make an ultimate determination such as blood alcohol content.

While a preferred embodiment has been set forth in detail above, those skilled in the art who have reviewed the present disclosure will readily appreciate that other embodiments can be realized within the scope of the invention. Therefore, the invention should be construed as limited only by the appended claims.

What is claimed is:

1. A method for detecting an analyte in a region of interest, the method comprising:
(a) illuminating the region of interest using light from a light source whose output wavelength is sensitive to temperature;
(b) during step (a), varying the temperature to which the light source is exposed so as to vary the output wavelength;
(c) during steps (a) and (b), receiving a portion of the light from the region of interest; and
(d) performing spectroscopy on the portion of the light received in step (c) to detect the analyte.

2. The method of claim 1, wherein the analyte is ethanol.

3. The method of claim 2, wherein the region of interest is a part of a living human body.

4. The method of claim 1, wherein the light source comprises an LED.

5. The method of claim 1, wherein:
step (a) comprises directing a portion of the light away from the region of interest;
step (c) comprises detecting the portion of the light directed away from the region of interest; and
step (d) comprises performing normalization in the spectroscopy, using the portion of the light directed away from the region of interest.

6. A system for detecting an analyte in a region of interest, the system comprising:
a light source for illuminating the region of interest, the light source having an output wavelength that is sensitive to temperature;
a temperature source for varying the temperature to which the light source is exposed so as to vary the output wavelength; and
a spectroscopic subsystem for receiving a portion of the light from the region of interest and performing spectroscopy on the portion of the light to detect the analyte.

7. The system of claim 6, wherein the spectroscopic subsystem is configured such that the analyte is ethanol.

8. The system of claim 8, wherein the spectroscopic subsystem is configured such that the region of interest is a part of a living human body.

9. The system of claim 6, wherein the light source comprises an LED.

10. The system of claim 6, wherein:
the light source is configured to direct a portion of the light away from the region of interest; and
the spectroscopic subsystem is configured to detect the portion of the light directed away from the region of interest and to perform normalization in the spectroscopy, using the portion of the light directed away from the region of interest.
